(19) United States
(12) Patent Application Publication
(10) Pub. No.: US 2009/0012376 A1
(43) Pub. Date: Jan. 8, 2009

(54) Continuous Analyte Monitor and Method of Using Same

(75) Inventor: Michael Agus, Boston, MA (US)

Correspondence Address: KOHN & ASSOCIATES, PLLC, 30500 NORTHWESTERN HWY, SUITE 410, FARMINGTON HILLS, MI, 48334, US

(73) Assignee: CHILDREN'S MEDICAL CENTER CORPORATION, Boston, MA (US)

(21) Appl. No.: 10/575,694

(22) PCT Filed: Nov. 3, 2004
(86) PCT No.: PCT/US2004/036565

§ 371 Date: Sep. 18, 2008

Related U.S. Application Data
(60) Provisional application No. 60/517,135, filed on Nov. 3, 2003.

(51) Int. Cl. A61B 5/1473; A61M 37/00

(52) U.S. Cl. 600/309; 604/66

(57) ABSTRACT

The use of an analyte-monitoring device for continuously monitoring analytes within a bodily fluid bypass flow path. A method of monitoring analytes in a patient by continuously monitoring analytes present in a bodily fluid of that patient within the bodily fluid bypass flow path is provided.

### BACKGROUND OF THE INVENTION

1. Field of the Invention

The present invention generally relates to a blood analyte testing system. More specifically, the present invention relates to a method and apparatus for use in continuously monitoring blood analyte concentrations.

2. Description of the Related Art

Recent studies have demonstrated a striking clinical benefit associated with strict glycemic control in the critically ill adult patient. Data from these studies show that maintaining serum glucose levels within a tight normal range using intravenous insulin infusions can reduce in-hospital mortality and common morbidities. While it remains unclear whether the clinical benefit is associated with reduced serum glucose levels or increased exogenous administration of insulin, the implementation and maintenance of strict glycemic control for the critically ill adult patient is rapidly becoming the nationwide standard of care.

The study of improved glycemic control in children, however, still requires advances in modem glucose monitoring techniques. At present, the available methods for measuring the concentration of blood glucose have relied upon portable bedside glucose measuring devices and laboratory glucose analysis. While both methods are practical in the intensive care unit setting, bedside devices may be inaccurate at the extremes of blood glucose concentration and may miss subtle trends in blood glucose while standard laboratory techniques require a substantial amount of blood, take a considerable amount of time for measurement and communication, and may be expensive. Thus, the trend towards strict glycemic control in the intensive care unit has created a need for glucose monitoring techniques that are both highly accurate and readily attainable.

The most critically ill neonate cared for in the intensive care unit, with respiratory or cardiac failure that has failed to respond to medical therapy, is supported with heart-lung bypass, or extracorporeal membrane oxygenation (ECMO). Due to their severity of illness, these patients are likely to benefit the most from glucose control, which can be most safely performed in the setting of continuous glucose monitoring. Because of the full anti-coagulation therapy that they must receive in order to prevent clotting in the circuit, they are not currently candidates for any glucose sensor to be implanted either internally or in their subcutaneous tissue. Thus, a sensor that can be implanted into the circuit itself, without requiring any exposure to an additional surgical, or even minimally invasive procedure, would be ideal in this population.

Initial work in the field of continuous glucose monitoring has focused upon the ambulatory patient with diabetes. To date, no attempts have been made to bring this evolving continuous glucose monitoring technology to the intensive care unit for use in a bypass circuit. Similarly, other specific patient populations of all ages (neonate, child, and adult) could benefit from the same technology, including those on ECMO in the intensive care unit, cardiopulmonary bypass in the operating room, continuous hemodialysis or hemofiltration, standard kidney hemodialysis, and liver dialysis.

The development of an integrated in vivo implantable glucose monitor was first reported by Wilkins and Atanasov (1995). The system utilizes glucose oxidase immobilized within a micro-bioreactor. The enzyme catalyzes the oxidation of beta-D-glucose by molecular oxygen to yield gluconolactone and hydrogen peroxide, with the concentration of glucose being proportional to the consumption of O₂ or the production of H₂O₂. Unfortunately, the presence of a glucose oxidase inhibitor molecule in the human bloodstream tended to offset proportionality constants, and made the device unsatisfactorily inaccurate for precise glucose monitoring and control (Gough et al., 1997).

Several nonspecific electrochemical sensors have also been investigated as potential in vivo glucose sensors (e.g., Yao et al., 1994; Larger et al., 1994), but problems including limited sensitivity, instability, and limited long-term reliability have prevented their wide-spread utilization (Patzer et al., 1995). According to Atanasov et al. (1997), continuously functioning implantable glucose biosensors with long-term stability have yet to be achieved.

Despite a significant miniaturization of biosensors during the past decade, they still require violation of the patients body, even if only minimally at the level of the skin.

It would therefore be useful to develop a method for continuously monitoring glucose concentrations of a patient without requiring the patient's body to be violated or blood to be drawn while overcoming the problems detailed above.

### SUMMARY OF THE INVENTION

According to the present invention, there is provided the use of an analyte-monitoring device for continuously monitoring analytes within a bodily fluid bypass flow path. A method of monitoring analytes in a patient by continuously monitoring analytes present in a bodily fluid of that patient within the bodily fluid bypass flow path is provided.

### BRIEF DESCRIPTION OF THE DRAWINGS

Other advantages of the present invention are readily appreciated as the same becomes better understood by reference to the following detailed description when considered in connection with the accompanying drawings, wherein:

FIG. 1 is a graph showing partial pressure in oxygen in VGMS sensors for performing the method of the present invention versus Bayer Rapidlab 860;

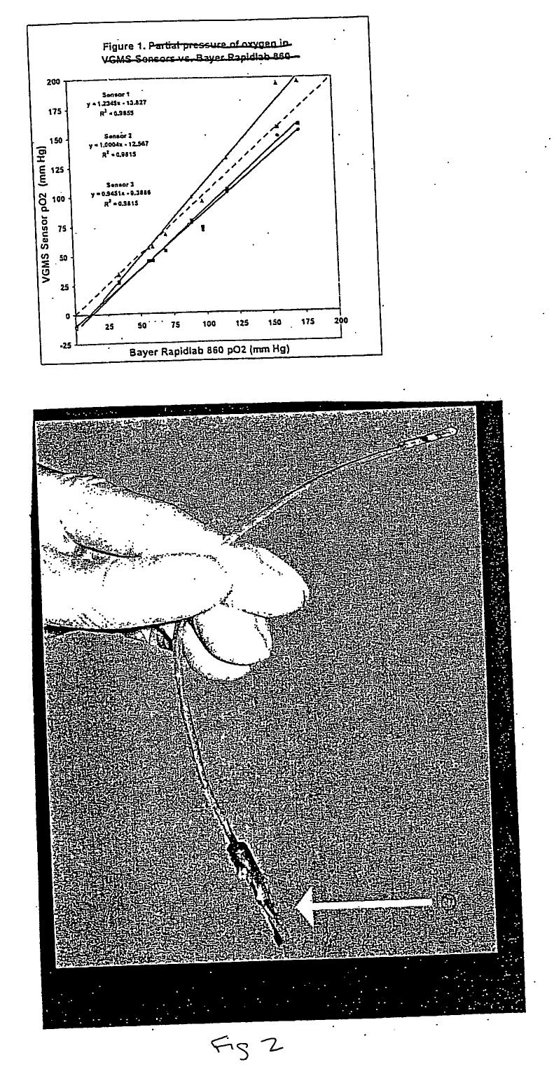

FIG. 2 is a photograph showing one embodiment for performing the method of the present invention of the present invention;

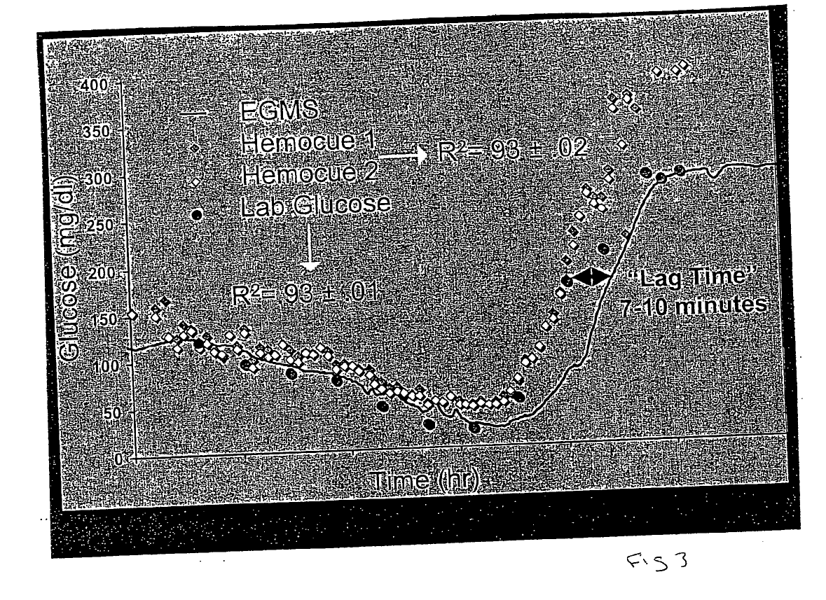

FIG. 3 is a graph showing the comparison of glucose measurements;

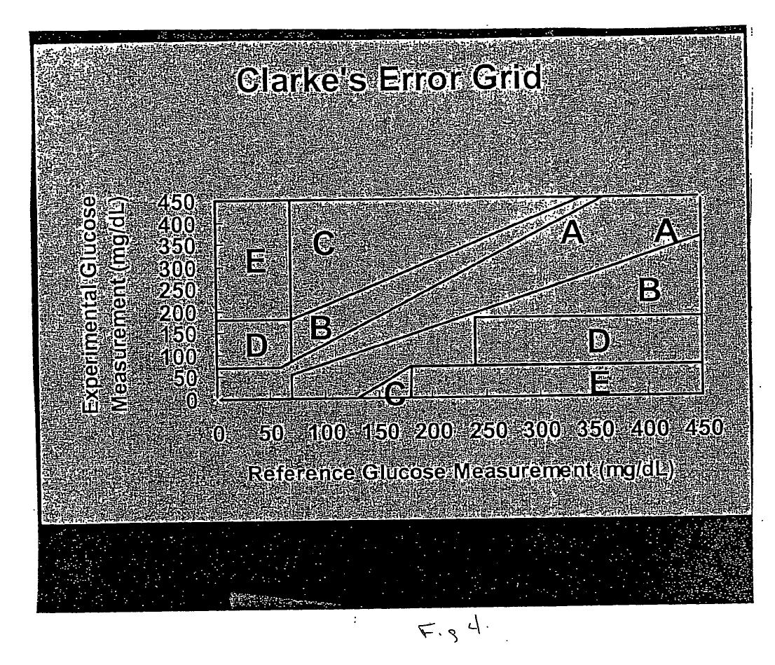

FIG. 4 is a graph showing the experimental glucose measurement versus the reference glucose measurement on a Clarke Error Grid;

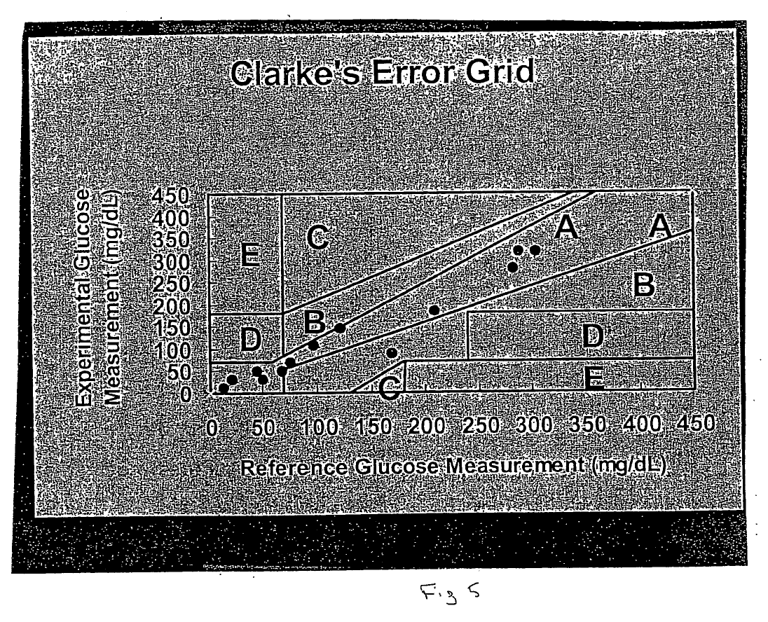

FIG. 5 is a graph showing the experimental glucose measurement versus the reference glucose measurement on a Clarke Error Grid for EGMS versus lab glucose;

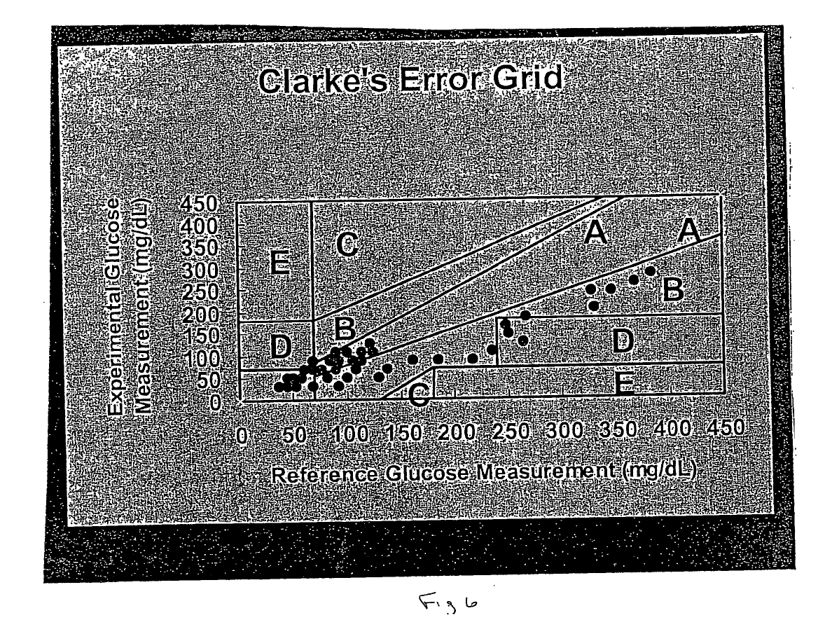

FIG. 6 is a graph showing the experimental glucose measurement versus the reference glucose measurement on a Clarke Error Grid for EGMS versus hemocue; and

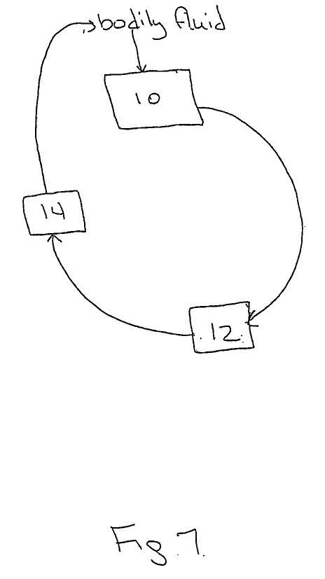

FIG. 7 is a block diagram of the system for performing the method of the present invention.

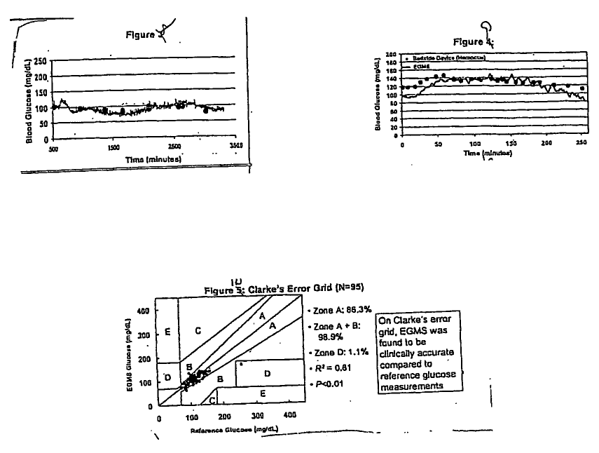

### DETAILED DESCRIPTION OF THE PRESENT INVENTION

Generally, the present invention provides a system for continuously monitoring analyte concentrations of a patient. More specifically, the present invention provides a device that can be used while the patient is attached to a bypass circuit for monitoring analyte concentrations and providing a mechanism to respond to such concentrations when they are outside of set norms.

The system of the present invention includes a sensor 10 operably connected to a monitor 12 for monitoring the concentrations of a desired analyte in a patient. The sensor 10 is placed within a bypass flow path, such as a blood exchanging circuit, and continuously senses the concentrations of the analyte in blood. The data obtained from the sensor 10 is transmitted to a monitor 12. The monitor 12 can either alert health care professionals of the concentrations of the analyte or can be further connected to a responding device 14 that can administer necessary compounds to return the analyte concentrations to normal ranges as are known to those of skill in the art.

The term “analyte” as used herein is intended to include, but is not limited HIV, viruses, medication concentrations, cholesterol, hormones, ammonia, fluids, glucose, electrolytes (e.g., sodium, potassium, chloride) minerals (e.g., calcium, phosphate, magnesium), lactate and other monitorable analytes known to those of skill in the art.

The term “sensor” as used herein is intended to include, but is not limited to, any device that is able to monitor and quantify a desired analyte. The analyte sensor 10 can be modified to alter the size, shape and orientation of the electrodes that come in contact with the interstitial fluid during analyte sensing. Examples of such sensors include, but are not limited to, electrochemical sensors capable of being used in vivo.

An “electrochemical sensor” is a device configured to monitor the presence and/or measure the concentration of an analyte in a sample via electrochemical oxidation and reduction reactions on the sensor 10 in a bypass flow path. These reactions are transduced to an electrical signal that can be correlated to an amount, concentration, or level of an analyte in the sample.

A “bypass flow path” is a path through which fluid flows during an operation or procedure. For example the bypass flow path can be an ECMO circuit, cardio-pulmonary bypass circuit, liver dialysis circuit, kidney dialysis circuit, or a continuous hemodialysis or hemofiltration circuit. The bypass can be a total bypass or a partial bypass. For a cardio-pulmonary total bypass, all the patient's systemic venous return blood is diverted from the right side of the heart into an extracorporeal circuit, emptying the chambers of the heart. The circuit includes a heart-lung machine that comprises a pumping function and an oxygenation function, completely taking over cardiopulmonary function for the patent, returning oxygenated blood to the aorta. In a partial bypass only a portion of the blood is diverted to the extracorporeal circuit. The remaining flow passes to the lungs and from the lungs through the coronary and systemic arterial circulation.

Electrochemical sensors are used to determine the concentrations of various analytes in testing samples such as fluids and dissolved solid materials. For instance, electrochemical sensors have been made for measuring glucose in human blood. This type of sensor has been used by diabetics and health care professionals for monitoring blood glucose concentrations. The sensors are usually used in conjunction with a meter, which measures light reflectance, if the strip is designed for photometric detection of a die, or which measures some electrical property, such as electrical current, if the strip is designed for detection of an electroactive compound.

Typically, electrochemical sensors are manufactured using an electrically insulating base upon which conductive inks such as carbon and silver are printed by screen printing to form conductive electrode tracks or thin strips of metal are unrolled to form the conductive electrode tracks. The electrodes are the sensing elements of the sensor generally referred to as a transducer. The electrodes are covered with a reagent layer comprising a hydrophilic polymer in combination with an oxidoreductase or a dehydrogenase enzyme specific for the analyte. Further, an insulating layer is mounted over a portion of the base and the electrodes.

Precision and accuracy of electrochemical measurements to a great extent rely on the reproducibility of the electrode surface area on a microscopic scale. Variations in the morphology of the electrode can result in very significant changes in the electrochemical signal readout. Screen-printing has made significant in-roads in the production of sensors for determining glucose. The wide use of screen-printing stems from the ability to mass-produce relatively inexpensive sensors. The use of metal strips unrolled from large rolls has also been employed to mass-produce such sensors.

The present invention provides an electrochemical sensor 10 for determining various analyte concentrations in a testing sample such as fluids and dissolved solid materials. The sensor 10 can be produced in large quantities using reliable and cost effective injection molding manufacturing methods. The present invention includes an injection molded plastic strip or body, at least two electrodes, an enzyme, and if desired, an electron transfer mediator. The body includes a cavity or reaction zone for receiving a fluid sample. The electrodes are at least partially embedded within the plastic body and extend into the reaction zone where they are exposed to a test sample. Also contained within the reaction zone is an enzyme capable of catalyzing a reaction involving a compound within the fluid sample.

The sensor 10 cooperates with an electronic meter capable of measuring the difference between the electrical properties of the electrically conductive electrodes within the device. The sensor 10 includes at least two, and preferably three, spaced apart electrically conductive electrodes, a body having two ends of insulative material molded about and housing the electrodes, means for connecting the meter to the housing, means for receiving a fluid sample, and means for treating one or more electrodes with one or more chemicals to change the electrical properties of the treated electrodes upon contact with the fluid sample. One end of the housing includes means for connecting the meter. The opposite end of the housing includes means for receiving the fluid sample. The means for connecting the meter is a plug formed in the housing exposing the electrodes outside the body.

The sensor 10 is molded and can be a single, unitary piece or two pieces. In the two-piece construction, an end cap is attached to the body. In the single piece construction, the body pivots about a hinge and connects onto itself. Protuberances formed in a portion of the body cooperate with troughs to ensure proper alignment.

A capillary inlet is constructed at one end of the sensor 10 to draw the fluid sample into the body upon contact with the fluid sample. The capillary inlet is molded into the end of the body and is in communications with a reaction zone. This reaction zone is a channel formed in the body about the electrodes and is adapted for reacting with the fluid drawn into the body by the capillary force. While the reaction zone can be formed above or below the electrodes, the preference has been to construct it above the electrodes. The capillary has a vent for relieving pressure.

As noted, the electrodes are molded into the plastic. In one embodiment, the electrodes are conductive wires. Alternatively, the electrodes can be constructed from a metal plate. The electrodes can be coated with a different conductive material to enhance their performance.

Apertures are formed in the body of the sensor 10 to permit the holding of the electrodes during the molding process. Apertures can also be formed in the body to chemically treat one or more electrodes in the reaction zone before or after the molding process. Adding chemicals (e.g., reagents with and without enzymes) changes the electrical properties of the treated electrodes upon contact with the fluid sample. In the preferred embodiment, the enzyme is applied to the outer surface of one of the electrodes. An antibody can also be applied to another of the electrodes. An electron mediator can further be applied to the outer surface of one or more of the electrodes.

In another embodiment made in accordance with the invention, the sensor 10 provides fill monitoring. Fluid drawn into the capillary inlet and the reaction zone contacts the edges of the electrodes, and upon reaching the lower end of the reaction zone, the area farthest from the capillary inlet, activates the meter. When the fluid comes in contact with the last electrode in the capillary space, it closes an open circuit in the electrochemical cell causing current to flow through the cell. The flow of current in the cell triggers the meter, signaling that the capillary chamber is filled with fluid. The vent could also be used for a visual monitor of fluid fill.

The method of making the sensor 10 includes the steps of positioning at least two spaced apart electrically conductive electrodes in a mold, before or after molding treating at least one of the electrodes with one or more chemicals to change the electrical properties of the treated electrode upon contact with a fluid sample, and molding a body of insulative material with two ends around the electrodes with one end having therein a device for receiving a fluid sample. The body is molded in two pieces, with a body and end cap for attaching to one another after the molding is completed, or in a single, unitary piece.

The chemical reaction most commonly used in enzyme coupled glucose sensors is the glucose oxidase mediated catalytic oxidation of glucose by atmospheric oxygen to produce gluconolactone and hydrogen peroxide (equation 1):

C₆H₁₂O₆+O₂+H₂O→C₆H₁₂O₇+H₂O₂ (1)

In the presence of excess oxygen, the quantity of hydrogen peroxide produced in this reaction will be a direct measure of the glucose concentration. The hydrogen peroxide is monitored by being reoxidized by an electrode (anode) maintained at a sufficient positive potential (equation 2):

H₂O₂-2e⁻→O₂+2H⁺ (2)

The glucose monitoring process is dependent upon the measurement of electrons removed from hydrogen peroxide in equation (2). The electrode is normally formed from a noble metal such as gold or platinum, or other metal known to those of skill in the art to function in accordance with the disclosure of the present invention.

Glucose sensors are used to measure glucose concentrations within a subject's body tissues. The glucose sensor 10 of the present invention can be used externally or internally as an implantable sensor. Accurate measurements of glucose concentrations in very low oxygen environments are obtainable with the glucose sensor 10 of the present invention. In order to achieve accurate measurements of glucose concentrations within the blood, the concentration of oxygen at the site of glucose oxidation must be greater than or equal to the glucose concentration at the site of glucose oxidation such that the glucose is the limiting factor in the oxidation reaction rather than the oxygen. To achieve and maintain this stoichiometric relationship at the site of glucose oxidation, the glucose concentration must be restricted and oxygen transport to the site of glucose oxidation must be enhanced.

Preferably, the analyte sensor 10 provided by the present invention includes a membrane system including an outer membrane and an enzyme-containing membrane, and an electrode. The enzyme-containing membrane is disposed between the outer membrane and the electrode.

The electrode can be any suitable electrode that is capable of monitoring and measuring hydrogen peroxide. Preferably, the electrode is a noble metal electrode, more preferably a platinum electrode. It is desirable that the surface of the electrode is maintained electroactive to maximize the effectiveness of the glucose sensor 10. Furthermore, it is desirable that the electrode does not change its sensitivity to hydrogen peroxide over time.

In operation, glucose and oxygen contained within the body tissues of a subject come into contact with the outer membrane of the glucose sensor 10. The outer membrane provides greater restriction to glucose than to oxygen and thus, reduces the concentration of glucose flowing through the outer membrane. The function of the outer membrane is to affect the concentrations of glucose and oxygen such that after the glucose and oxygen have passed through the outer membrane, the concentration of oxygen is preferably greater than or equal to the concentration of glucose. By doing so, the outer membrane establishes the stoichiometric relationship required for the glucose oxidation reaction.

After the stoichiometric relationship between the oxygen and the glucose has been established by the outer membrane, this stoichiometric relationship must be maintained at the sites of glucose oxidation, namely the enzymes contained within the enzyme-containing membrane. Maintaining this stoichiometric relationship at the enzymes is facilitated by the semi-interpenetrating polymer network and its enhancing effects on oxygen transport. Furthermore, the enzyme-containing membrane creates a path for the glucose in the glucose's attempt to pass through the membrane, however, the membrane does not restrict the flow of glucose to the enzymes. This added restrictive control on glucose and the enhanced oxygen transport to the enzymes, such that localized concentrations of oxygen are formed, ensures that the stoichiometric relationship is maintained at the enzymes. Therefore, at a particular enzyme, the concentration of oxygen at the enzyme is greater than or equal to the concentration of glucose at the enzyme. As a result of the stoichiometric relationship between oxygen and glucose at the enzymes, oxygen does not act as the limiting factor in the glucose oxidation reaction. Thus, the hydrogen peroxide generated during the glucose oxidation corresponds to the glucose present at the enzyme. Current flow representative of oxidation of hydrogen peroxide at the anode is measured relative to a reference electrode so that a complete circuit is formed. The reference electrode is commonly provided by a silver or silver/silver chloride electrode in electrical contact with the body fluids.

The outer membrane is preferably a polycarbonate but can be formed of any other suitable solid porous or permeable material. The outer membrane reduces the rate of mass transport of the glucose through the membrane and yet does not interfere with the rate of mass transport of the oxygen through the membrane. Thus, the outer membrane provides the restrictive control for the glucose. The outer membrane also prevents catalase, an enzyme that destroys hydrogen peroxide, and other large molecules from passing through the membrane. The pore size and thickness of the outer membrane are selected to ensure that the passage of glucose through the outer membrane is sufficiently hindered in comparison to the passage of oxygen. In general, the thicker the membrane and the smaller the pore size, the more the passage of glucose is hindered. In implantable glucose sensors, the outer membrane must be made from a suitable biocompatible material.

The glucose sensor 10 includes three electrodes (a working electrode, a counter electrode and a reference electrode). To optimize the electrochemistry of the glucose sensing reaction, it is preferred that the counter electrode is the largest electrode, the working electrode (i.e., the one with enzymes, or the like) is the next largest electrode and the reference electrode is the smallest electrode. Preferably, the counter electrode is as large as possible and consistent with sensor insertion requirements to minimize pain on insertion of the sensor 10 into the body of the user. For instance, the sensor 10 can be designed to fit within a 22-gauge needle. However, alternative embodiments can be sized to fit other gauge needles ranging from 18 to 30 gauges. In addition, altering the size of the working electrode affects the amount of enzyme that can be placed on the working electrode and affects the overall life of the glucose sensor 10. The analyte sensor 10 can use other types of sensors, such as chemical based, optical based, or the like. The sensors can also be sensors previously used intravascularly or subcutaneously.

The sensor 10 of the present invention is preferably in communication with an external monitoring device 12, which converts the readings or data from the sensor 10 into decipherable information. For example, the monitoring device 12 can convert the information into data that triggers an alarm indicating a problem with the glucose concentration concentrations, either too high or too low. Alternatively, the monitoring device 12 can convert the information into data that is transmitted to a responding device 14 that responds to the information. In other words, the monitoring device 12 can be in communication with a second device, a responding device 14, that, when notified of altered analyte concentrations, can provide an appropriate remedy, i.e. when measuring glucose the responding device, if a deviation from normal concentrations is detected, the responding device 14 injects either insulin or sugar.

The analyte monitor 12 also removes inconvenience by separating the complicated monitoring process electronics into two separate devices; a analyte monitor 12, which attaches to the analyte sensor 10; and a data processor, computer, communication station, or the like, which contains the software and programming instructions to download and evaluate data recorded by the glucose monitor. In addition, the use of multiple components (e.g., analyte monitor and data processor, computer, communication station, or the like) facilitates upgrades or replacements, since one module, or the other, can be modified or replaced without requiring complete replacement of the monitor system. Further, the use of multiple components can improve the economics of manufacturing, since some components may require replacement on a more frequent basis, sizing requirements can be different for each module, different assembly environment requirements, and modifications can be made without affecting the other components.

The software utilized in the present invention enables the system of the present invention to be automated. More specifically, the software enables the system to automatically detect, monitor, and adjust analyte concentrations within a patients bodily fluid. The software of the present invention creates a feedback loop such that when analyte concentrations detected by the sensor 10, and received by the monitor 12, are outside of the range of set norms, the software enables a responding device 14 to administer a compound or compounds to alter the analyte concentrations to reach normal concentrations.

In use, the analyte monitor 12, in this instance a glucose monitor, takes raw glucose sensor data, such as glucose data or the like, from the subcutaneous-glucose sensor 10 and assesses it during real-time and/or stores it for later download to the data processor, computer, communication station, or the like, which in turn analyzes, displays and logs the received glucose readings. Logged data can be analyzed further for detailed data analysis. In further embodiments, the glucose monitor system can be used in a hospital environment or the like. Still further embodiments of the present invention can include one or more buttons on the glucose monitor 12 to program the monitor 12, to record data and events for later analysis, correlation, or the like. In addition, the glucose monitor 12 can include an on/off button for compliance with safety standards and regulations to temporarily suspend transmissions or recording. The glucose monitor 12 can also be combined with other medical devices to combine other patient data through a common data network and telemetry system.

Communication can occur via a wireless network, a wired network, a modem, radio frequency, and any other connections known to those of skill in the art. Additionally, the device can include a mode that permits physician (or other medical practitioner) controlled programming of the second device, in response to the data obtained from the sensor 10, to the exclusion of the user. The device can also include a remote that includes a link to a computer to allow programming to initiate or alter available capabilities of the device. Also, the device can store patient infusion history and device activity.

The system of the present invention can be placed within in a variety of systems that filter or otherwise transport blood. For example, the system can be used in an ECMO circuit, cardiopulmonary bypass circuit, liver dialysis circuit, kidney dialysis circuit, or a continuous hemodialysis or hemofiltration circuit. The system of the present invention can be placed in vivo, ex vivo, or in vitro. In other words, the system can be placed within the bodily fluid flow path inside of the patient, outside of the patient's body but without removing fluid from the patient, or can be used in connection with a system that continuously removes a small amount of fluid from the patient for monitoring analyte concentrations.

A typical pediatric ECMO circuit is composed of numerous components that include a venous reservoir, a roller (or impeller) pump, a membrane oxygenator, a heat exchanger, polyvinylchloride connecting tubing, and connectors. Blood is passively drained by gravity from the venous circulation using a siphon height of 100 cm or more into a collapsible bladder that acts as a compliant reservoir. The bladder has a proximity switch attached to its top surface that acts to regulate the roller pump by turning it off when the bladder deflates. This mechanism limits the maximum suction applied to the patient to the hydrostatic pressure created by the siphon. Blood then passes through an occlusive roller pump and is forced at flow rates ranging from 120 to 170 ml/min/kg through a membrane oxygenator, such as a Kolobow U.S. Pat. No. 3,969,240. Oxygenated and CO₂ cleared blood is then returned via a heat exchanger at body temperature back to the patient's circulation.

Extracorporeal perfusion is used for the most part in cardiac bypass surgery. In a total bypass, all of the patient's systemic venous return blood is diverted from the right side of the heart into an extracorporeal circuit, emptying the chambers of the heart. The circuit includes a heart-lung machine that comprises a pumping function and an oxygenation function, completely taking over cardiopulmonary function for the patient, returning oxygenated blood to the aorta. In a partial bypass only a portion of the blood is diverted to the extracorporeal circuit. The remaining flow passes to the lungs and from the lungs through the coronary and systemic arterial circulation. Partial bypass usually is temporarily used following total bypass surgery to slowly give the heart work to do, slowly decreasing flow through the heart-lung machine, until the heart is weaned from assist and can fully take over its pumping role.

Some procedures using blood pumps in extracorporeal circulation do not include an oxygenation function. These include cardiac assist procedures. In these procedures, the blood pump provides higher systemic blood pressure and more blood flow than can be provided by a failing heart. A “fem-fem” (femoral vein to femoral artery) circuit is commonly used. Cardiac assist is also sometimes used if, after open heart surgery, the left side of the heart responsible for pumping to the body oxygenated blood returned from the lungs does not resume its pumping role despite attempts at weaning. If other assist circulatory devices are unsuccessful, the left heart can be bypassed to the aorta by cannulation of the left atrium, with the blood that has been oxygenated by the lungs being withdrawn through the cannula and pumped to the aorta extracorporeally without extracorporeal oxygenation.

Extracorporeal circulation is used in “extracorporeal life support,” also called “extracorporeal membrane oxygenation,” known by their respective acronyms of “ECLS” or “ECMO”, for simplicity herein called only ECMO. As opposed to the more conventional extracorporeal circulation in substitution or assist of the cardiac function, ECMO connotes the application of such support to supply oxygenation where the native lungs are compromised. This is especially useful for neonates, including premature birth babies, whose life is threatened because their diseased lungs cannot provide adequate gas exchange, and/or their pulmonary blood flow is compromised due to constriction of the pulmonary vessels (pulmonary hypertension), or in a neonate or older child or adult whose heart is failing and is unable to sustain normal circulation and perfusion. Another use is resuscitated drowning victims or other patients with severe infection whose lungs are damaged and unable to supply adequate oxygenation without restorative healing, a condition known as acute respiratory distress syndrome (ARDS). The extracorporeal circulation provides oxygenated blood to the patient's lungs under the impetus of the patients native heart and gives time to allow healing of the lungs to occur until the lungs can take over oxygenation. In excess of 1,000 ECMO procedures are conducted annually in the United States.

The basic components of the ECMO system include a blood pump, a membrane oxygenator, a countercurrent heat exchanger to warm the blood, and a control module. In the typical extracorporeal circuit, deoxygenated blood drains by gravity into the circuit and flows into the venous reservoir, usually placed 25 to 30 inches below the plane of the great veins. If the oxygenator is a bubble type, the reservoir is incorporated into an oxygen-blood mixing chamber. In any case, the reservoir is placed upstream to the pump, for reasons amplified below, to prevent negative pressure in the inlet line. A water heat exchanger is used for the perfusate to control body temperature. Blood filters are used to trap particulate and gaseous emboli. The arterial cannula is usually placed in the ascending aorta but can be placed downstream In the arterial system where the vessel is large enough to accommodate the necessary flow.

The blood pump is the “heart” of the extracorporeal perfusion circuit. In general, extracorporeal circulation systems use either an occlusive compression peristaltic roller pump or a non-ompressive centrifugal pump. Both produce flow rates from less than one up to several liters per minute, thus can apply well to adult usage requirements.

The basic roller pump consists of two rollers, 180 degrees apart. The rollers rotate in a circle through a half circular raceway. A length of flexible tubing, having an inner diameter of between ¼ and ⅝ inch, is placed between the rollers and the raceway. The rollers, rotating in a circular movement, compress the tubing against the raceway, squeezing the blood ahead of the rollers. The rollers are set to almost completely occlude the tubing, and operate essentially as a positive displacement pump, each passage of a roller through the raceway pumping the entire volume of the fluid contained in the tubing segment between the rollers.

Numerous surgical services limit the complications derived from roller pumps by using non-compressive pump centrifugal pumps. Centrifugal pumps rapidly rotate an impeller in a stationary blood compartment. The impeller can be a series of blades that push the blood forward, or it can be nested concentric cones of increasing diameter to propel the blood forward by centrifugal force. In nested concentric cone centrifugal pumps, flow is a function of outflow line pressure, so these pumps have advantage over the bladed impeller centrifugal pumps and the roller pumps, namely, the nested concentric cone centrifugal pumps do not produce high back pressures when the downstream tubing is temporarily obstructed. Roller and centrifugal pump extracorporeal systems were designed for open-heart surgery on adults. Roller and centrifugal pump extracorporeal systems produce flow rates on the order of several liters per minute, responding to requirements of adult usage. These systems are applied for children through the miniaturization of the same designs.

U.S. Pat. No. 3,784,323, to Sausse, discloses a pump that is available for pumping volume so the output is controlled as a function of inlet pressure. The Sausse (Rhone-Poulenc) pump stretches a distensible silicon tubing of an ovoid or elliptical cross section and shape memory compliance longitudinally around pin rollers mounted 120 degrees apart on a rotating wheel, the tubing being held in place below the wheel by connectors retained in a notched fixed base. This tubing, herein called a “header” tubing, is not compressed against a raceway (as for a roller pump), but is held in tension across the rollers, restricting the lumen of the header tubing across the rollers. This segments the header tubing into portions defined by leading and trailing adjacent rollers. The rotation of the wheel moves fluid captured between adjacent rollers in the direction of the rotation. The material and thickness of the wall of the header tubing are selected so the tubing between the rollers expands or collapses as a function of pump inlet pressure (available venous return). Collapse of the tube restricts the flow rate of the liquid as a function of the pump inlet pressure. If the venous supply decreases and inlet pressure drops, flow rate lessens, even though the pump speed is unchanged, and the inlet line remains filled. Consequently, no dangerously low negative pressures can occur. When outflow obstruction occurs, the liquid blocked from flowing forward can back flow, so the pump feeds nothing forward to over pressurize and burst the return line. Instead, the back flow accumulates in the stretched header tubing, which distends or expands to accommodate the additional volume. When the obstruction is released, blood flows downstream propelled by the increased stroke volume of the distended header tubing. The header tubing stretched over the rollers therefore functions as a built-in capacitance reservoir, eliminating the need for the reservoirs that are required for roller and centrifugal pumps. Accordingly, the pump is self-regulating and is remarkably safe.

The flow rate of the Sausse type pump can be considered as substitute cardiac output and pump suction volume as diverted venous return. The compliance of the header tube allows its volume to increase under the action of the suction pressure. The volume is evacuated in the form of a bolus, and its evacuation causes the tube to regain a flat shape capable for being refilled. This compliance provides a level of security that, as mentioned, is similar to that of a reservoir.

The stretched header tubing is located so that the hydrostatic head of the inlet line (venous return), relative to the lift height for the outlet line (downstream pressure, which determines maximum flow rate), does not render the pump body insensitive to changes in upstream pressure. Tension imposed on the stretched header tubing is a function of tubing wall thickness and elasticity, roller diameter, delivery pressure, pump speed, and flow rate.

Because the Sausse type pump does not require a venous reservoir, priming volumes for an extracorporeal circuit can be much smaller than with circuits using roller or centrifugal pumps. Hence, there is less dilution of a patient's blood; so patient hematocrit can be maintained higher without red blood cell augmentation. This makes the Sausse type pump well adapted for employment in extracorporeal systems for infants. Use of the Sausse type pump for ECMO in normal birth weight infants and babies has been described by Chevalier, J. Y., Durandy Y., Basses A. et al., “Preliminary Report: Extracorporeal Lung Support for Neonatal Acute Respiratory Failure,” Lancet 1990, vol. 335, pp 1364-1366; and by Trittenwein, G., Furst, G., Golej et al. “Preoperative ECMO in Congenital Cyanotic Heart Disease Using the AREC System,” Ann. Thorac Surg 1997, vol. 63, pp 1298-1302.

The device of the present invention can be used in conjunction with Continuous Renal Replacement Therapy (CRRT), for use in treating patients suffering from excess fluid overload and acute renal failure. In the acute setting, CRRT has been performed previously using standard methods of continuous hemodialysis and continuous hemofiltration. Continuous veno-venous hemofiltrabon (CWH) has been used to reduce the complications associated with such issues as hemodynamic instability and need for arterial access.

Renal replacement therapy performs two primary functions: ultrafiltration (removal of water from blood plasma), and solute clearance (removal of different molecular weight substances from blood plasma). The filter, also called hemofilter or “dialyzer”, can be set up to perform either or both of these functions simultaneously, with or without fluid replacement, accounting for the various modes of renal replacement therapy. “Clearance” is the term used to describe the removal of substances, both normal and waste product, from the blood.

Ultrafiltration is the convective transfer of fluid out of the plasma compartment through pores in the membrane. The pores filter electrolytes and small and middle sized molecules (up to 20,000 to 30,000 daltons) from the blood plasma. The ultrafiltrate output from the filtration pores is similar to plasma, but without the plasma proteins or cellular components. Importantly, since the concentration of small solutes is the same in the ultrafiltrate as in the plasma, no clearance is obtained, but fluid volume is removed.

Dialysis is the diffusive transfer of small solutes out of a blood plasma compartment by diffusion across the membrane itself. It occurs as a result of a concentration gradient, with diffusion occurring from the compartment with higher concentration (typically the blood compartment) to the compartment with lower concentration (typically the dialysate compartment). Since the concentration of solutes in the plasma decreases, clearance is obtained, but fluid may not be removed. However, ultrafiltration can be combined with dialysis.

Hemofiltration is the combination of ultrafiltration, and fluid replacement typically in much larger volumes than needed for fluid control. The replacement fluid contains electrolytes, but not other small molecules. Since the net effect of replacing fluid without small solutes and ultrafiltration of fluid with small solutes results in net removal of small solutes, clearance is obtained.

The device of the present invention can be used in conjunction with kidney hemodialysis. Hemodialysis is a process by which excess waste products and water are removed from the blood. This process requires an access to the patients blood stream and the use of a hemodialysis machine. An access is a specially created vein in the arm known as arterio-venous (AV) fistula. In hemodialysis, the blood channels through plastic tubings (blood lines), driven by the force of a roller pump which moves the blood to the dialyzer which Is a bundle of hollow fibres made up from semi-permeable membrane. Here the exchange (diffusion) takes place from blood to the dialysis solution (dialysate) and vice versa. The dialysate has a salt composition similar to blood but without any waste products. Usually one dialysis session takes about 4 hours to complete and patient requires dialysis 3 times a week.

The device of the present invention can be used in conjunction with liver dialysis. Liver dialysis is similar to kidney dialysis, but several differences exist. In the dialyzer, the blood is passed through a chamber instead of through many small tubes. The membrane separating the blood from the dialysate functions like a diaphragm, pumping the blood and dialysate in and out of the dialyzer alternately resulting in an average transmembrane pressure of 100-200 mm Hg. These pressure changes along with ports at the top and bottom of the blood chamber are used to move the blood through the circuit, avoiding the use of roller pumps and their associated hemolysis. The volume of blood within the circuit varies from 200-250 cc depending on whether the system is in inflow or outflow.

The dialysate itself also differs from that used in kidney hemodialysis. Instead of a buffered aqueous solution, liver dialysis uses a mixture of sorbents including powdered activated charcoal, cation exchange resin, salts, a buffering agent, and macromolecular wetting substances. Although the dialysate is cycled repeatedly through the system, the charcoal provides a surface area of approximately 300,000 m2 providing almost constant clearance rates during the entire 4-6 hour treatment. As in kidney dialysis, substances such as glucose and electrolytes can be added to the dialysate to prevent their removal from the patient's blood.

ECMO in neonates is also complicated by the low circulating blood volume of the patients and by the difficulty of obtaining vascular access. The total blood volume of a neonate is generally appreciably less than the priming volume of the typical ECMO circuit. A volume of donor blood equivalent to several total exchange transfusions is thus required simply to prime the circuit.

The preferred blood oxygenator assembly of the present invention comprises a membrane oxygenator device that is approximately three inches in length (not including inlet/outlet connectors) and approximately two inches in diameter. The preferred membrane oxygenator device is sized to operate effectively at fluid rates that do not exceed the flow capacity of the blood vessel into which the perfusion fluid is being infused. For coronary angioplasty procedures, the membrane oxygenator device is preferably sized and constructed to operate effectively with fluid (e.g. blood) flow there through at 30 to 60 ml/min. and preferably at about 45 ml/min., as such flow rate can generally be accommodated by the coronary artery into which the perfusion fluid (e.g. blood) is being infused. The device incorporates a blood inlet, a blood outlet, a gas inlet, and a gas outlet. A blood supply tube is fluidly attached to a blood withdrawal device, such as a needle or other percutaneously insertable conduit positioned with the patient's vasculature. A pump can be positioned on tube so as to pump blood from the patient's vasculature, through blood supply tube and into the membrane oxygenator component. A blood return tube is connected to the blood outlet of the membrane oxygenator component. The blood return tube is attachable to the proximal end of a balloon angioplasty catheter having a perfusion supply lumen extending there through, so as to provide a flow of hemoperfusion blood from the membrane oxygenator component, through the perfusion supply lumen and out of the distal portion of the catheter, distal to the occlusive angioplasty balloon.

The system of the present invention can be fit within a fitting on the proximal end for mating to a syringe. The fitting is often a Luer fitting, which describes generally the male-female shapes of the syringe and needle hub, respectively. When the Luer fitting includes means such as threads for locking the male and female parts together, the fitting is known as a Luer-lock tip. The Luer-lock fitting is a standard fitting in the medical field, often having a single thread having nominally three turns about the longitudinal axis of the syringe. The Luer-lock fitting is well suited for administering agents such as drugs through common hypodermic needle lengths. Alternative fittings can also be used to house the system of the present invention.

The system of the present invention is preferably used during a surgical operation on an infant In use, the sensor 10 is either placed within a fitting in a bodily fluid bypass flow during the surgery or the sensor 10 is included in within the fitting during the manufacturing of the fitting. The sensor 10 is operably connected to the monitor 14 of the present invention in a manner disclosed above, preferably via a wireless connection. The sensor 10 can continuously monitor analyte concentrations within the patient. The data obtained by the sensor 10 is transmitted to the monitor 12. The monitor can further be in communication with a responding device 14. The responding device 14 can administer to the patient a compound, or compounds, which adjusts the analyte concentrations within the patient such that the concentrations are within a range of set norms. The system can be controlled manually or via software.

The present invention can also be used to calibrate a second monitor that is coupled to a sensor set to provide continuous data recording of readings of glucose levels from a sensor for a period of time. Preferably, the monitor is worn by the user and is connected to a surface mounted sensor set that is attached to a user's body by an electrically conductive cable The sensor can use any type of sensors known to be useful for monitoring the analyte. Examples of such sensors include, but are not limited to, chemical based, optical based, or other similar sensors. The sensors can be placed on an external surface of the skin or placed below the skin layer in the subcutaneous tissue of the user for detecting analyte concentrations.

The method of the present invention can be used to calibrate a monitor because any device used for performing the method of the present invention will be monitoring, in a bodily fluid bypass flow path, analyte levels in vivo. The data obtained as a result of this can be used to calibrate a monitor capable of monitoring analyte levels outside of the bodily fluid bypass flow path. While the calibration can be performed manually, it is preferred that the method be automated. Software can be used to automatically calibrate a monitor based on the information obtained by the sensor used for performing the method of the present invention.

The present invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

In light of the reduced mortality associate with strict glycemic control and insulin infusion in critically ill patients, there is a vital need for reliable real-time continuous glucose monitoring In the intensive care unit. An intravascular device that measures serum glucose concentration on a continuous basis has been developed: Extracorporeal Glucose Monitoring System (EGMS, Medtronic Minimed, Northridge, Calif.). The device is an electrochemical sensor 10 in a 7.5 French silicone catheter that measures glucose concentration using a glucose oxidase reaction. In this study, the first use of continuous intravascular glucose monitoring in an extracorporeal membrane oxygenator (ECMO) circuit is described.

### Methods

In a bench study, a neonatal ECMO circuit was primed with saline and three EGMS sensors were inserted at the pre-bladder, pre-oxygenator, and post-heat exchanger locations. The saline was then displaced per ECMO protocol with human blood products. Circuit blood glucose concentrations were altered by saline dilution and dextrose Infusion to create hypo-, normo-, and hyperglycemic conditions. Temperature was kept constant at 36° C. Flow was maintained at 300 cc/min. Serum glucose concentrations were measured as follows: (1) at one-minute intervals by each EGMS device; (2) at five-minute intervals using a bedside glucose dehydrogenase reaction in duplicate (HemoCue B-Glucose Analyzer, Angelholm, Sweden); and (3) at thirty-minute intervals using a glucose oxidase reaction in the clinical laboratory (Bayer Rapidlab® 860, Tarrytown, N.Y.). Independent comparisons using Clarke's error grid were made with Bayer 860 and HemoCue measurements to analyze the accuracy of the device.

### Results

All three continuous glucose sensors recorded real-time data throughout the experiment without interruption. There was no significant pressure drop across the sensors at any of the three circuit locations. EGMS glucose measurements closely correlated with Bayer 860 (R²=0.933±0.01) and with HemoCue (R²=0.928±0.016) glucose measurements. Using Clarke's error grid of analysis with Bayer 860 as the reference value, 89.6% of EGMS readings were within sector A (clinically correct) and 100% were within sectors A+B (clinically correct+clinically acceptable). With HemoCue as reference, 59.9% were within sector A, 94.4% within sectors A+B and 5.6% were in sector D (clinically unacceptable). EGMS glucose values demonstrated an approximate 7-10 minute lag while circuit blood glucose concentrations were rapidly changing.

### Conclusions

The results of this pilot study suggest that the EGMS continuous intravascular glucose-monitoring device is a reliable tool for measuring blood glucose in the extracorporeal circuit. The disagreement of the sensor with a small subset of HemoCue values warrants further investigation. Potential applications of this technology include intensive glucose monitoring in patients on ECMO support, cardiopulmonary bypass, and renal replacement therapy (CWH, CAVH). Further research is required to explore the functionality of this glucose monitoring system in various clinical settings.

### Example 2

Continuous monitoring of glucose as well as partial pressure of oxygen (pO₂) has become a desirable tool in the care of critically ill patients. A continuous glucose and pO₂-monitoring device was designed for insertion into an extracorporeal membrane oxygenation (ECMO) circuit: Extracorporeal Glucose Monitoring System (EGMS, Medtronic Minimed, Northridge, Calif.). The device is an electrochemical sensor that functions as an amperometric Clarke electrode housed in a 7.5 French silicone catheter. It is connected by a wire to a transmitter, which sends glucose and pO₂ data each minute to a nearby computer.

### Methods

The EGMS device was placed into a saline-primed neonatal ECMO circuit using a 0.8 m² silicone membrane oxygenator at three positions: pre-bladder, pre-membrane, and post-heat exchanger. The catheter was introduced into the ECMO circuit via a hemostatic valve. The saline was then displaced per ECMO protocol with human blood products. Temperature was maintained at 36° C. 1. To evaluate pO₂ accuracy, gas admixtures were delivered to the membrane oxygenator to maintain pO₂ at 30, 60, 90, and 200 mm Hg. Each pO₂ was maintained for 40 minutes and blood gases were sampled every 20 minutes (Bayer Rapidlab® 860, Tarrytown, N.Y.). 2. To assess impedance to blood flow, pressure transducers were positioned pre- and post- each catheter insertion site and pressure measurements were recorded at five flow rates 300, 500, 700, 1,000, and 1,200 cc/min at circuit hours 1, 24, and 48.

### Results

1. The EGMS sensor pO₂ values were strongly correlated with those measured by the Bayer 860: R²=0.983±0.001 (See Figure). There was no discemable lag time associated with even rapid changes in pO₂.
2. There were no pre- or post- pressure changes at any of the tested flow rates, at any of the sensor sites up to 48 hours.

### Conclusions

The EGMS continuous glucose and pO₂ sensor produced accurate pO₂ data in a real-time fashion throughout a broad range of blood oxygen concentration. It appears to be a safe device to insert into even the smallest caliber ECMO circuit without compromising blood flow across it. The sensor can be used for monitoring blood glucose and pO₂ in ECMO and other extracorporeal applications.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. An in vivo analyte-monitoring device for use in continuously monitoring at least one analyte the presence of an analyte within a bodily fluid bypass flow path.

2. The use of the analyte-monitoring device according to claim 1, wherein said sensor means provides continuous real-time monitoring.

3. The use of the analyte-monitoring device according to claim 1, wherein said sensor means provides periodic real-time monitoring.

4. The use of the analyte-monitoring device according to claim 1, wherein said sensor means is an electrochemical sensor.

5. The use of the analyte-monitoring device according to claim 1, wherein said sensor means can monitor at least one analyte selected from the group consisting essentially of HIV, viruses, medication concentrations, cholesterol, hormones, fluids, glucose, electrolytes, lactate and other monitorable analytes.

6. The use of the analyte-monitoring device according to claim 1, wherein said sensor means quantitates the amount of the analyte detected.

7. The use of the analyte-monitoring device according to claim 1, further including monitoring means operatively connected to said sensor for monitoring the amount of analytes detected by said sensor means and comparing the detected amounts to set norms.

8. The use of the analyte-monitoring device according to claim 7, further including responding means operatively communicating with said monitoring means for responding to detected amounts of analytes outside of the norms in order to bring the amounts detected back into the range of the set norms.

9. The use of the analyte-monitoring device according to claim 8, wherein said responding means includes administration means for administering to the patient at least one compound for bringing the amount of the analyte in the patient back within the set norms.

10. The use of the analyte-monitoring device according to claim 1, wherein said bypass flow path is selected from the group consisting essentially of an extracorporeal membrane oxygenation circuit, cardiopulmonary bypass circuit, a continuous hemodialysis circuit, a continuous hemofiltration circuit, a kidney dialysis circuit and a liver dialysis circuit.

11. A method of monitoring analytes in a patient by continuously monitoring analytes present in a bodily fluid of the patient within a bodily fluid bypass flow path.

12. The method according to claim 11, further including the step of monitoring the amount or concentration of analytes detected and comparing the amounts detected to set norms.

13. The method according to claim 12, further including the step of responding to the detected amount of analytes outside of set norms to bring the amount detected back to a range within the set norms.

14. The method according to claim 13, wherein said responding step is further defined as administering to the patient at least one compound and bringing the amount of analytes detected back into the range of the set norms.

15. The method according to claim 14, wherein said responding step includes administering to the patient insulin.

16. The method according to claim 12, wherein said monitoring step includes monitoring changes in the amount of analytes.
